(19) United States
(12) Patent Application Publication
(10) Pub. No.: US 2009/0215196 A1
(43) Pub. Date: Aug. 27, 2009

(54) Sequential Method

(75) Inventors: Bo Ek, Bjorklinge (SE); Mats Holmquist, Sollentuna (SE); Karolina Osterlund, Uppsala (SE); Mats Inganas, Uppsala (SE)

Correspondence Address: FULBRIGHT & JAWORSKI, LLP, 1301 MCKINNEY, SUITE 5100, HOUSTON, TX, 77010-3095, US

(73) Assignee: GYROS Patent AB, Uppsala (SE)

(21) Appl. No.: 11/572,316

(22) PCT Filed: Jul. 15, 2005
(86) PCT No.: PCT/SE2005/001154

§ 371 Date: Apr. 15, 2009

Related U.S. Application Data
(60) Provisional application No. 60/588,712, filed on Jul. 16, 2004.

(30) Foreign Application Priority Data
Jul. 16, 2004 (SE) 04/01888-3

(51) Int. Cl. G01N 33/566

(52) U.S. Cl. 436/501

(57) ABSTRACT

A method for determination of an analyte in an original liquid sample by performing an inhibition affinity assay in a microchannel structure of a microfluidic device. The main characteristic feature is the steps of: (i) providing the microfluidic device in a form where the microchannel structure comprises a reaction microcavity which a) an inlet end, and b) contains a solid phase with binding sites (BS) for an analyte or an analyte-related entity, (ii) providing a liquid sample (1) containing the analyte or the analyte-related entity within the microchannel structure at the inlet end of the reaction microcavity, and flowing sample (1) through the reaction microcavity under conditions such that the analyte or the analyte-related entity is captured by BS leaving a portion of BS unoccupied (free BS), (iii) providing a liquid sample (2) which contains an analytically detectable analogue of the analyte or of the analyte-related entity, and flowing sample (2) through said reaction microcavity to capture the analogue by free BS, and (iv) measuring the amount of analyte analogue captured in step (iii) and relating this amount to the amount of analyte in an original sample.

### TECHNICAL FIELD

The invention is a microfluidic heterogeneous inhibition affinity assay for the determination of an analyte in a liquid sample. The assay is carried out in a microchannel structure of a microfluidic device that typically contains a plurality of microchannel structures. The determination is quantitative in the sense that one determines the presence or absence of the analyte including also the amount, if so desired.

### TECHNICAL BACKGROUND AND OBJECTS OF THE INVENTION

A heterogeneous inhibition affinity assay typically comprises an affinity reaction of an analyte and an analyte analogue with an affinity counterpart (=analyte counterpart) that provides a binding site BS that is common for both the analyte and the analyte analogue. The analyte and the analyte analogue thus are capable of mutually inhibiting their binding to BS. In this kind of assays the affinity reaction may be carried out in one step with all three reactants present at the same time, or as a two-step procedure during which the counterpart is reacted with the other two reactants one at a time. Either the analyte analogue or the counterpart may be pre-immobilized to a solid phase while the other one is dissolved in the liquid used and analytically detectable (measurable). The use of a solid phase means that it is possible to separately measure the amount of the detectable reactant on the solid phase and/or remaining in dissolved form after the affinity reaction. Subsequent to the measurement the value obtained is typically related to the amount of analyte or analyte-related entity participating in the reaction and/or being present in an original sample.

The term “competitive affinity assay” is used synonymously to “inhibition affinity assay” in the context of the invention.

Reactions between a dissolved reactant and a reactant immobilized on a solid phase in inhibition affinity assays has previously been carried out during non-flow or flow conditions and with or without “stirring”.

When designing microfluidic inhibition affinity assays a key feature has been mixing of liquid aliquots, e.g. for diluting purposes and/or for accomplishing aliquots containing the appropriate combination of reactants. Mixing of μL-aliquots, such as nl-aliquots, of liquids in microfluidic systems is relatively complicated and typically requires microchannel structures that have separate mixing functions. If mixing could be avoided simpler microfluidic structures could be used and microfluidic devices with a higher density of microfluidic structures designed. Potentially the same microchannel structure could be used for inhibition and non-inhibition assay protocols, which would result in a more versatile use of microfluidic devices containing this kind of microchannel structures.

There is therefore a need for novel microfluidic inhibition affinity assays of the type discussed above that do not require mixing.

WO 01074438 (Gyros AB), WO 04083108 Gyros AB), WO 04083109 Gyros AB), WO 04106296 (Gyros AB), SE 0403030-0 and US Provisional filed Dec. 9, 2004 (“Microfluidic assays and Microfluidic devices”) (Gyros AB) disclose various kinds of microfluidic heterogeneous inhibition affinity assays.

Chromatographic affinity assays including various competitive and non-competitive variants are described in Clarke et al (“Development of sandwich HPLC microcolumns for analayte adsoerption on the millisecond time scale”, Anal. Chem. 73 (2001) 1366-1373); Clarke et al (“Analysis of free drug fractions by ultrafast immunoaffinity chromatography”, Anal Chem. 73 (2001) 2157-2164); Hage et al (“Theory of a sequential addition competitive binding immunoassay based on high-performance immunoaffinity chromatography”, Anal. Chem. 65 (1993) 1622-1630); Hage et al (“Development of a theoretical model for chromatographic-based competitive binding immunoassays with simultaneous injection of sample and label”, Anal. Chem. 71 (1999) 2965-2975); Nelson et al (Chromatographic competitive binding immunoassays: a comparison of the sequential and simultaneous injection method”, Biomed. Chrom. 17 (2003) 188-200).

The measured response from a labelled reactant used in a inhibition affinity assay depends on a) the ratio between the amount of analyte and the amount of the counterpart, b) the ratio between the amount of analyte analogue and the amount of counterpart, and c) the flow rate during adsorption to the solid phase, and d) affinity between analyte/analyte analogue and the counterpart. Optimal responses are normally obtained when similar amounts of analyte and analyte analogues are bound to the analyte counterpart, i.e. the molar ratio between analyte and analyte analogue bound to the counterpart should be in the range of 0.1-10, such as 0.2-5 or 0.5-2. There is a general need for improvements that secure high chances for quickly obtaining optimal responses for liquid samples in which the analyte may be present within a wide concentration range.

There are often problems in reaching desired limits of detection, precisions (coefficient of variation, CV), dynamic ranges, signal to noise levels, recoveries, diagnostic specificities and sensitivities, sensitivities (measured as slope in response curves) etc in heterogeneous inhibition affinity assays. These problems are typically rendered more severe when carrying out the assay reactions under flow conditions and/or without letting the reactions go to equilibrium and in particular applies to the capture of the analyte by BS on the solid phase. It is therefore a general desire and goal for heterogeneous inhibition microfluidic affinity assays to accomplish acceptable levels with respect to these characteristics when one or more of the above-mentioned assay reactions are carried out under flow conditions and/or without need of complete capturing. In other words achieving e.g. a) limits of detection ≦10⁻⁶ M, such as ≦10⁻⁹ M or ≦10⁻¹² M or ≦10⁻¹³ M or ≦10⁻¹⁴ M or ≦10⁻¹⁵ M or ≦10⁻¹⁶ M, b) dynamic ranges that are more than two, three, four, five or more orders of magnitude, c) precisions (CV) within ±20%, such as within ±10% or within ±5% or within ±3%, d) recoveries ≧70% such as ≧80% or ≧90% or ≧95% or around 100% or more for the innovative method.

A particular object is to increase the dynamic range of the inventive method while maintaining the sensitivity (slope in response graph) and perform the capture of the analyte or an analyte-related entity by BS on the solid phase under flow conditions without the need of complete capture of the analyte or of an analyte-related entity that is present in liquid sample 1.

All patent applications and issued patents cited herein are hereby incorporated in their entirety by reference.

### DRAWINGS

FIG. 1 illustrates the microchannel structures of one of the microfluidic devices used in the experimental part.

FIGS. 2-6 represent the results obtained in examples 1-5.

### THE INVENTION

The invention is a method for the determination of an analyte in an original liquid sample by performing a inhibition affinity assay in a microchannel structure (101a-h) of a microfluidic device. The method is characterized in comprising the steps of:
(i) providing the microfluidic device in a form where the microchannel structure (101a-h) comprises a reaction microcavity (104a-h) which a) has one or more inlets, and b) contains a solid phase that exhibits binding sites (BS) that are capable of affinity binding to the analyte or an analyte-related entity,
(ii) providing a liquid sample 1 containing the analyte or the analyte-related entity within the microchannel structure (101a-h) at an inlet of the reaction microcavity (104a-h), and flowing sample 1 through the reaction microcavity (104a-h) under conditions such that the analyte or the analyte-related entity is captured by BS in an amount related to the amount of analyte or analyte-related entity of sample 1 and leaving a portion of BS unoccupied (=free BS),
(iii) providing a liquid sample 2 which contains an analytically detectable analogue of the analyte or of the analyte-related entity at an inlet of the reaction microcavity (104a-h), and flowing sample 2 through said reaction microcavity (104a-h) under conditions such that the analyte analogue is captured by the free BS in an amount that is related to the amount of BS that has become occupied during step (ii), and
(iv) measuring the amount of analyte analogue bound to the solid phase in step (iii) and relating this amount to the amount of analyte in the original sample.
   The term “affinity” in affinity counterpart, affinity assay etc includes a sufficient specificity for the purpose.

Step (ii) may divided in two, three or more repetitive substeps each of which comprises providing a portion of liquid sample 1 at an inlet of the reaction microcavity and flowing the portion through the microcavity to capture the analyte or analyte-related entity present in the portion on the solid phase. The portions are preferably of the same size/volume. This variant represents an easy way to increase the actual amount of analyte or analyte-related entity loaded on the solid phase and in particular applies if there is a risk that the concentration of the analyte or an analyte-related entity in an original sample introduced into the microchannel structure is too low to give an optimal response level in the assay.

### Liquid Sample 1, Original Liquid Sample, Analyte, and Analyte-Related Entity

The analyte and the analyte-related entity are typically bio-organic molecules in the sense that they exhibit one or more structures selected among
a) amino acid structures including protein structures such as peptide structures such as poly and oligopeptide structures, and including mimetics and chemically modified forms of these structures etc;
b) carbohydrate structures, including mimetics and chemically modified forms of these structures, etc;
c) nucleotide structures including nucleic acid structure, and mimetics and chemically modified variants of these nucleotide structures, etc; and
d) lipid structures such as steroid structures, triglyceride structures, etc, and including mimetics and chemically modified forms of these structures;
e) hormone structures, such as steroid structures, phytohormone structures, peptide structures etc.

The analyte and/or the analyte-related entity may also exhibit one or more functional groups selected among e.g. chelate/chelating groups, carboxy groups, amino groups, amido groups, phosphono groups, phospates, ether groups etc, which groups may or may not be part of a structure according to (a)-(e) above. The analyte and/or the analyte-related entity may be a hapten or an antigen in which case the binding site (BS) on the solid phase typically is part of the antigen- or hapten-binding part of an antibody molecule. A haptenic/antigenic structure is typically part of a larger molecule for instance selected among infectious agents (bacteria, algae, fungi, viruses, prions, moulds, parasites etc), drugs, autoantigens, allergens, synthetic or native immunogens that are capable of provoking humoral immune responses that are used in the manufacture of antigen/hapten specific antibody reagents, etc. The analyte and/or the analyte-related entity may be naturally occurring or man-made, for instance by being a chemically or recombinantly modified variant of a basic molecule exhibiting a peptide structure. The analyte may for instance be a member of a library of recombinantly produced members each of which exposes a tag structure that is capable of binding to the same binding site BS on the solid phase. A typical example is a peptide exhibiting a hapten structure such as a sequence of two or more histidine residues in which case the binding site on the solid phase may be part of the anti-hapten antibody such as an anti-his tag antibody, or a metal chelate.

In preferred variants the analyte or analyte-related entity is a non-antibody entity, i.e. it does not utilize the antigen-binding site of an antibody for binding to BS during step (ii). Liquid sample 1 derives from an original sample and contains the analyte or an analyte-related entity where the latter has been obtained by transformation of the original sample to liquid sample 1 containing the analyte or the analyte-related entity. The amount of analyte in the original sample and the amount of analyte or of the analyte-related entity in liquid sample 1 are related to each other. The transformation may take place fully or partly within the microfluidic device. Transformation within a microchannel structure typically includes transportation and/or various processing steps. Typical processing steps that are carried out within a microchannel structure (101a-h) are selected among volume-defining or metering, diluting, mixing, purifying, removing of particulate material such as cells, etc. Volume-defining is particularly important in preferred variants of the invention which means that then the preferred microchannel structure (101a-h) provided in step (i) comprises an inlet port that is associated with a volume-defining unit and that a liquid sample containing the analyte or an analyte-related entity is introduced via this port whereafter an aliquot of the sample introduced is defined in the volume-defining unit and subsequently transported downstream in the microchannel structure (101a-h) to provide liquid sample 1 at the inlet of the reaction microcavity (104a-h). The preferred volume-defining units are based on the overflow principle to fill up a volume-metering microcavity that in turn preferably comprises a constriction at the junction between an overflow microconduit and a metering microcavity. Details about the volume-defining unit are given elsewhere in this specification.

If not otherwise suggested by the particular context, the term “analyte” in this specification includes an original analyte as well as various analyte-related entities. The term “original sample” primarily will refer to the sample containing the analyte that is determined by the use of the measured value obtained in step (iii).

The original sample containing the analyte as well as liquid sample 1 and any intermediary forms that are formed during the transformation typically are biological fluids in the sense that they contain a compound exhibiting a structure or functional group of the kinds indicated above for the analyte or the analyte-related entity. In a more narrow sense the term biological fluid means a fluid which contains this kind of compounds and derives from a fluid the composition of which at least partially has been determined by living or dead biological material. In this restricted meaning biological fluids include cell culture supernatants, tissue homogenates, blood and various blood fractions such as serum or plasma, lachrymal fluid, regurgitated fluid, urine, sweat, semen, cerebrospinal fluid, gastric juice, saliva, lymph, etc as well as various liquid preparations containing a bio-organic compound as discussed above and deriving from these kinds of fluid. Liquid samples containing the analyte or the analyte-related entity typically derives from a vertebrate body fluid of the kinds discussed above. Typical vertebrates are mammals, avians, amphibians, reptiles etc. Typical mammals are whales, humans, mice, rats, guinea pig, horses, cows, pigs, dogs, cats etc. Typical avians are hens, canaries, budgerigars etc. A special class of vertebrates are those that are used in farming or as pets or are found in zoological gardens.

### Liquid Sample 2 and the Analytically Detectable Analogue

The analytically detectable analogue is capable of competing with the analyte or with the analyte-related entity about binding to the binding site BS. This means that the detetable analyte analogue and the analyte or the analyte-related entity exhibit a part structure (moiety 1) that is identical or for other reasons is capable of affinity binding to the binding site BS. The detectable analogue in addition comprises a structure (moiety 2, detectable group) that confers detectability to the analogue, i.e. the analogue can be measurably distinguished from other reactants on the solid phase after step (iii). The analyte and/or the analyte-related entity may in addition contain structures that are not present on the detectable analogue. The analyte may for instance be a conjugate between a protein and a tag that is capable of affinity binding to the binding site BS on the solid phase while the detectable analyte analogue may be a conjugate between a detectable structure and the tag without need for containing the protein structure that is present in the analyte.

The detectable analogue may exhibit structures selected from the same structures and functional groups as the analyte.

The detectable analyte analogue is typically provided in step (iii) in an amount that is in excess of the amount of free BS after step (ii) or the amount of BS provided in step (i). If the amount of detectable analogue is in excess, such as in an unlimited amount, it is important to secure that the detectable analogue does not replace analyte captured to the solid phase in step (ii) to an extent that destroys the relationship between the amount of analyte in the original sample/liquid sample 1 and captured analyte and/or free BS created in step (ii). This does not exclude that also a subcessive (deficient) amount, such as ≧50% or ≧75% or ≧90% of the amount of BS provided in step (i), can be used as long as the amount is capable of being captured by the solid phase in step (iii) in an amount that reflects the amount of analyte or analyte-related entity in liquid sample 1.

Moiety 2 of the detectable analogue typically comprises a group that can be analytically detected and quantified. Signal-generating groups and affinity groups are typical examples of detectable groups. A signal-generating group may be selected among radiation emitting or radiation absorbing groups and groups that in other ways interfere with a given radiation. Particular signal-generating groups are enzymatically active groups including enzymes, cofactors, substrates, coenzymes etc; groups containing particular isotopes such as radioactive or non-radioactive isotopes; fluorescent groups including fluorogenic groups; luminescent group including chemiluminescent groups, bioluminescent groups etc; metal-containing groups including metal ion containing groups etc. Typical affinity based detectable groups may be selected among the individual members of the immobilizing affinity pairs discussed below for introducing BS on the solid phase, with the proviso that an affinity based detectable group should not be capable of affinity binding during the method to a member of an immobilizing binding pair if such a pair has been used for the pre-introduction of BS on the solid phase.

Liquid sample 2 that is provided at the inlet of the reaction microcavity (104a-h) typically derives from a liquid sample containing the detectable analogue. This liquid sample is typically introduced via an inlet port and transported downstream to provide liquid sample 2 at the desired position close to the inlet end of the reaction microcavity (104a-h). The liquid sample may be processed during the transport for instance by one or more process steps selected among volume-definiting, mixing, diluting etc. Alternatively the analogue may be pre-dispensed and present in dry form within the microchannel structure where it is reconstituted by a liquid sample that is introduced via an inlet port whereafter the reconstituted sample is transported and possibly processed to provide liquid sample 2 at the inlet of the reaction microcavity (104a-h). As for the analyte and the analyte-related entity the liquid sample used for providing liquid sample 2 is preferably introduced via an inlet port that is associated with a volume-defining unit where an aliquot of an introduced sample is volume-defined and transported downstream to provide liquid sample 2 at an inlet of the reaction microcavity. The preferences are the same as for the original sample and liquid sample 1.

The Microchannel Structure (101a-h) Including the Reaction Microcavity (104a-h), The Immobilized Binding Site Bs and the Solid Phase Material.

The microchannel structure (101a-h) is part of a microfluidic device as described elsewhere in this specification and typically contains the microfluidic functions required for carrying out all the steps of an assay protocol that are intended to be carried out within the mcrofluidic device.

The reaction microcavity (104a-h) is defined as the part of a microchannel structure (101a-h) where the solid phase carrying the binding site BS is present. The upstream/inlet end of the reaction microcavity (104a-h) coincides with the corresponding end of the solid phase. This also applies to the downstream/outlet ends. The reaction microcavity (104a-h) may be a part of a larger chamber in which other solid phases may be placed upstream and/or downstream of the solid phase that contains BS. These other solid phases differ from the BS-solid phase in containing binding sites that are counterparts to other analytes (BS″, BS′″ etc and An″, An′″ etc) or are devoid of binding sites. It follows that these other solid phases define other reaction microcavities within the same chamber. Other parts of the microchannel structure (101a-h) may contain other reaction microcavities and chambers. The inlet end of a reaction microcavity may comprise one or more inlets.

The reaction microcavity (104a-h) is typically in the microformat, i.e. has at least one cross-sectional dimension that is ≦1 000 μm, such as ≦500 μm or ≦200 μm (depth and/or width). The smallest cross-sectional dimension is typically ≧5 μm such as ≧25 μm or ≧50 μm. The total volume of the reaction microcavity (104a-h) is typically in the nL-range, such as ≦5 000 nL, or 1 000 nL or ≦500 nL ≦100 nL or ≦50 nL or ≦25 nL.

The reaction microcavity (104a-h) typically has a length that is within the range of 1-100,000 μm, such as ≧10 μm or ≧50 μm or 100 μm or ≧400 μm, and/or ≦50 000 μm or ≦10 000 μm or ≦5 000 μm or ≦2 500 μm or ≦1 000 μm.

The solid phase may be in the form of porous bed, i.e. a porous monolithic bed or a bed of packed particles that may be porous or non-porous. Alternatively, the solid phase may be an inner wall of the reaction microcavity (104a-h). A monolithic bed may be in the form of a porous membrane or a porous plug.

The term “porous particles” has the same meaning as in WO 02075312 (Gyros AB).

Suitable particles are spherical or spheroidal (beaded), or non-spherical. Appropriate mean diameters for particles are typically found in the interval of 1-100 μm with preference for mean diameters that are ≧5 μm, such as ≧10 μm or ≧15 μm and/or ≦50 μm. Also smaller particles can be used, for instance with mean diameters down to 0.1 μm. Diameters refer to the “hydrodynamic” diameters. Particles to be used may be monodisperse (monosized) or polydisperse (polysized) in the same meaning as in WO 02075312 (Gyros AB).

The base material of a solid phase may be made of inorganic and/or organic material each of which may or may not be polymeric. Typical inorganic materials comprise glass. Typical organic materials comprise organic polymers. Polymeric materials comprise inorganic polymers, such as glass and silicone rubber, and organic polymers of synthetic or biological origin (biopolymers). The term “biopolymer” includes semi-synthetic polymers in which there is a polymer backbone derived from a native biopolymer. Appropriate synthetic organic polymers are typically cross-linked and often obtained by the polymerisation of monomers comprising polymerisable carbon-carbon double bonds. Examples of suitable monomers are hydroxy alkyl acrylates, for instance 2-hydroxyalkyl acrylates such as hydroxyethyl acrylates, and corresponding methacrylates, acryl amides and methacrylamides, vinyl and styryl ethers, alkene substituted polyhydroxy polymers, styrene, etc. Typical biopolymers in most cases exhibit carbohydrate structure, e.g. agarose, dextran, starch etc.

A suitable porous bed is typically wettable (hydrophilic) in the sense that water is spread by capillary force all throughout the bed when one end of the bed is in contact with excess water (absorption). This also means that the inner surfaces of a wettable bed that is in contact with an aqueous liquid medium during step (ii) shall expose a plurality of polar functional groups which each has a heteroatom selected among oxygen and nitrogen, for instance. Appropriate functional groups can be selected among hydroxy groups, ethylene oxide groups (—X—[CH₂CH₂O—]ₙ where n is an integer >1 and X is nitrogen or oxygen), amino groups, amide groups, ester groups, carboxy groups, sulphone groups etc. For solid phase materials in particle form this means that at least the outer surfaces of the particles have to exhibit polar functional groups. Similar material, wettabilities and functional groups etc also apply to solid phases in the form of inner walls.

The amount of BS provided in step (i) for a particular analyte is determined by the flow conditions used for transportation of the liquid sample through the reaction microcavity (104a-h) and the amount of the analyte or analyte-related entity provided in step (ii). After liquid sample 1 has passed through the reaction microcavity (104a-h) there must remain a measurable amount of free BS that uniquely reflects the amount of analyte or analyte-related entity provided in step (ii). In order to accomplish this the molar amount of analyte or analyte-related entity provided in step (ii) relative to the molar amount of BS provided in step (i) may be selected as a excessive or subsessive amount whereafter the flow rate for liquid sample 1 through the reaction microcavity (104a-h) is adapted accordingly to achieve free BS as given above. The molar amount of analyte or analyte-related entity may thus be selected within the range of 0.1% to 10 000% of the molar amount of BS, such as ≧1% or ≧10% and/or ≦1 000% or ≦500%. A lower flow rate will lead to a higher yield in the capturing than a higher flow rate. Sufficiently low flow rates leads to complete capturing of the analyte and selection of molar amounts of analyte or analyte-related entity provided in step (ii) that preferably are be in the interval 1-99%, such as 5-95%, or 10-90% or 20-80% or 25-75% of the molar amount of BS provided in step (i). At an increased flow rate, the efficiency in the capturing will go down permitting also excesses of analyte or analyte-related entity, for instance above 100% such as above 200% or even higher.

Experimental tests are typically required for determining optimal binding capacity for analyte, amount of BS on the solid phase, kind of solid phase etc. The amount of BS given above refers to the amount of BS that initially is available for capture of the analyte or the analyte-related entity.

The binding site BS is part of an analyte affinity counterpart that is immobilized to the solid phase. This affinity counterpart including its binding site for the analyte may in principle comprise structures and functional groups selected from the same structures/functional groups as discussed above for the analyte. In the preferred variants the binding site BS is part of an anti-analyte antibody molecule. In this context the term antibody comprises intact antibody molecules as well as various antigen/hapten-binding fragments and derivatives thereof including recombinantly produced forms such as single chain antibodies. It follows that in the case the binding site is specific for a structure that is recurring on many different analyte molecules the same solid phase can be used for individually quantifying each of these analyte molecules. A typically example is various isoforms of a protein. Another typical example is a library of organic molecules, such as proteins or peptides, in which the identical tag has been introduced, for instance by recombinant techniques, on the individual members, e.g. a peptide tag such as a histidine tag or some other haptenic tag.

The technique used for the introduction of BS, on the solid phase is typically according to one or both of two main routes:
a) immobilization of the analyte counterpart, and
b) building the analyte counterpart stepwise on the solid phase (solid phase synthesis of an immobilised analyte counterpart that exhibits BS).

Both routes are commonly known in the field. The linkage to the solid phase material may be via covalent bonds, affinity bonds (for instance biospecific affinity bonds), physical adsorption, electrostatic bonds etc.

Alternative (a) typically makes the use of an immobilizing pair of two reactive structures—one RSₛₚ on the solid phase and another one R_{cp} on the analyte counterpart. These two reactive structures are mutually reactive with each other to the formation of a bond that immobilizes the analyte counterpart to the solid phase and resists undesired cleavage when performing steps (ii) and subsequent steps of the innovative method. RSₛₚ is typically pre-introduced on the solid phase material before step (i) or is inherently present on the solid phase material. The formation of the immobilizing bond is carried out either before or during step (i).

Covalent immobilization means that the reaction of RSₛₚ with RS_{cp} leads to covalent attachment of the counterpart/BS to the solid phase. RSₛₚ and RS_{cp} are typically selected among electrophilic and nucleophilic groups. Examples of groups that may be used are amino groups and other groups comprising substituted or unsubstituted —NH₂, carboxy groups (—COOH/—COO⁻), hydroxy groups, thiol groups, disulfide groups, carbonyl groups (keto, aldehyde), groups containing carbon-carbon double and triple bonds, haloalkyl groups. Particular important in this context are reactive forms (activated forms) of this kind of groups, e.g. reactive esters, reactive amides or imides, alkene and alkyne groups to which one or more carbonyls are directly attached (α-β unsaturated carbonyls), haloalkyl to which a carbonyl group is directly attacked α-halo carbonyl) etc. Free radical reactions may also be used.

Immobilization via affinity bonds utilizes an immobilizing affinity pair in which one of the members (immobilized ligand, L=RSₛₚ) is firmly attached to the solid phase material. The other member (immobilizing binder, B=RS_{cp}) is used as a conjugate (immobilizing conjugate) comprising binder B and BS (where BS is part of an analyte counterpart, such as an anti-analyte antibody if the analyte is an antigen or a hapten). The pair is typically selected such that it does not interfere with the desired affinity reaction of step (ii) between the analyte and its affinity counterpart which means that the same immobilizing pair may be used for the immobilization of a range of different analyte counterparts. In other words the immobilizing affinity pair including the affinity ligand L and the affinity binder B is generic. Examples of immobilizing affinity pairs are a) biotin-binding compounds such as streptavidin, avidin, neutravidin, anti-biotin antibodies etc, and biotin, b) anti-hapten antibodies and the corresponding haptens or antigens, c) IMAC groups (immobilized metal affinity chelates) and an amino acid sequence containing histidyl and/or cysteinyl and/or phosphorylated amino acid residues (i.e. an IMAC motif), d) anti-species specific antibodies and Igs from the corresponding specie, e) class/subclass-specific antibodies and Igs from the corresponding class, f) Igs and Ig-binding proteins originating from microorganisms etc. The term Ig (immunoglobulin) includes Ig-fragments and derivatives containing Ig-specific structures/determinants and corresponding proteins in non-mammal vertebrates, e.g. Ig-fragments and Ig-derivatives that comprise species- or class/subclass unique determinants. The term antibody includes various antibody forms as discussed elsewhere in this specification.

The term “conjugate” primarily refers to covalent conjugates, such as chemical conjugates and recombinantly produced conjugates. The term also includes so-called native conjugates, i.e. a native affinity reactant which exhibits two binding sites that are spaced apart from each other with affinity directed towards two different molecular entities. Native conjugates thus include an antigen, which has physically separated antigenic determinants, an antibody molecule which has a species and/or class-specific determinant in one part of the molecule and an antigen/hapten-binding site in another part.

Preferred immobilizing affinity pairs (L and B) typically have affinity constants (K_{L--B}=[L][B]/[L--B]) that are at most equal to the corresponding affinity constant for streptavidin and biotin, or ≦10¹ times or ≦10² times or ≦10³ times larger than this latter affinity constant. This typically will mean affinity constants that roughly are ≦10⁻¹³ mole/L, ≦10⁻¹² mole/L, ≦10⁻¹ mole/L and ≦10⁻¹⁰ mole/L, respectively. The preference is to select L and B among biotin-binding compounds and streptavidin-binding compounds, respectively, or vice versa. These affinity constant ranges refer to values obtained by a biosensor (surface plasmon resonance) from Biacore (Uppsala, Sweden), i.e. with the ligand L immobilized to a dextran-coated gold surface.

The binding capacity of the solid phase for RS_{cp} can be measured as the amount of RSₛₚ in mole divided by volume. With this measure suitable binding capacities will typically be found within the interval of 0.001-3000 pmole, such as 0.01-300 pmole, divided by nL of solid phase in bed form saturated with liquid. For instance, if 0.1 pmole streptavidin per nL has been immobilized this corresponds 0.4 pmole biotin-binding sites per nL. The conversion factor four is because a streptavidin molecule has four binding sites for biotin.

Binding capacity can also be measured as actual binding capacity for RS_{cp}, i.e. mole active RS_{cp}-binding sites divided by volume of the solid phase that contains RSₛₚ (bed form and saturated with liquid such as water). This kind of binding capacity will depend on the immobilization technique, the pore sizes of the solid phase, the size of the entity to be immobilized, the material and design of the solid phase etc. Ideally the same ranges apply for the actual binding capacity as for the total amount of binding sites (preceding paragraph).

Measurements of actual binding capacities can be carried out according to principles well known in the field. Se for instance WO 2004083109 (Gyros AB).

In the case the solid phase is an inner wall of the reaction microcavity, the volume of the solid phase is taken as the volume of the reaction microcavity.

A reactive structure RSₛₚ and/or BS may be introduced on the solid phase while the solid phase is placed in the reaction microcavity (104a-h) or in a batch mode with the solid phase placed outside the microchannel structure. (101a-h) If a batch mode is used for the introduction of RSₛₚ the solid phase may subsequently be transferred to the reaction microcavity (104a-h) of a microchannel structure (101a-h) where it may be further transformed to exhibit BS. The latter transformation may also take place in a batch mode outside the flow path. Alternatively both steps are carried out while the solid phase is placed in the reaction microcavity.

In the case the reaction mixture used for introduction of the analyte counterpart according to the preceding paragraph contains a) the analyte counterpart in a form that exposes RS_{cp} and a nonsense reactant that exhibits a reactive structure RSₙₛ that also reacts with RSₛₚ in the same manner as RS_{cp}, or b) a subcessive (deficient) amount of the analyte counterpart that exhibits RS_{cp}, one can easily control the capacity of the final solid phase for capturing the analyte by varying the amount of the counterpart and/or of the nonsense reactant. A solid phase obtained for these reactant combinations will contain at least two kinds of structures that derive from RSₛₚ—one kind immobilizes the analyte counterpart while the other one is not linked to the analyte counterpart, i.e. immobilizes the nonsense reactant or is the remaining group RSₛₚ. The term “nonsense reactant” means a reactant that does not interfere with the desired affinity reaction between the analyte counterpart and the analyte/analyte-related entity/detectable analyte analogue and includes affinity counterparts that have specific affinity for other analytes (An″,An′″etc). The term thus includes that several different such reactants are present simultaneously. In this kind of solid phase the molar ratio between the amount of immobilizing binding structures for the analyte counterpart and the sum of the amount of immobilizing structure for the nonsense reactant and the amount of remaining structures RSₛₚ may be in the interval ≧0.01, such as ≧0.10 or ≧0.20 or ≧0.40 or ≧0.50 or ≧0.60 or ≧0.70, and/or ≦0.99, such as ≦0.90 or ≦0.80 or ≦0.70 or ≦0.60 or ≦0.50 or ≦0.40 or ≦0.30. Although the ratio between the corresponding reactive groups in the reaction mixture may be selected within the same intervals the ratio as such may be different. See also U.S. provisional Ser. No. 60/685,554 (“Bridging assay and a flow path with a versatile solid phase”).

In preferred variants the immobilization method above is part of step (i) and comprises the steps of: a) providing the reaction microcavity with a solid phase that is in a form exhibiting the reactive structure RSₛₚ but no BS, and b) providing a liquid sample 3 containing the counterpart at an inlet of the reaction microcavity (104a-h) and flowing sample 3 through the microcavity under conditions such that the counterpart will become immobilized to the solid phase at least in a zone close to the inlet used. Liquid sample 3 may contain a nonsense reactant or a deficient amount of reactants exhibiting RS_{cp} and/or RSₙₛ. In preferred variants the microchannel structure contains an inlet port that is associated downstream of which there is a volume-defining unit as discussed elsewhere in this specification. A liquid sample containing the reactants to be immobilized via the immobilizing pair is preferably introduced via this inlet port and further transported and/or processed in the structure to provide liquid sample 3 at the reaction microcavity.

Immobilizing affinity pairs are preferred as RSₛₚ and RS_{cp} for the introduction of the analyte counterpart.

Flow conditions and capturing of analyte (step (i)) and the analyte analogue (step (ii))

The term “flow conditions” means that the analyte or the analyte-related entity in step (ii) and the detectable analogue in step (iii) are presented to the BS on the solid phase while the liquid in which these reactants are present is continuously flowing through the reaction microcavity (104a-h)/solid phase. In other words the affinity complex between the analyte/analyte-related entity and the pre-immobilized BS (step (ii)) and between the detectable analogue and the pre-immobilized BS (step (iii)) is formed during flow conditions. The flow rate used may or may not provide diffusion limiting conditions for the affinity reaction.

The appropriate flow rate through the porous bed depends on a number of factors:
a) the pre-immobilized analyte counterpart (that exhibits the binding site BS);
b) the kind of analyte or analyte-related entity, e.g. size and form, affinity constant of the analyte/analyte-related entity and the detectable analogue;
c) the dimensions of the reaction cavity (volume, length etc),
d) the kind of solid phase (the solid phase material, porosity, bed or coated inner wall etc); and
e) etc.

Typically the flow rate should give a residence time of ≧0.010 seconds such as ≧0.050 sec or ≧0.1 sec for liquid sample 1, i.e. the liquid aliquot containing the analyte or the analyte-related entity and passing through the reaction microcavity (104a-h). The upper limit for residence time is typically below 2 hours such as below 1 hour. Illustrative flow rates are within 0.001-10 000 nL/sec, such as 0.01-1 000 nL/sec or 0.01-100 nL/sec or 0.1-10 nL/sec. These flow rate intervals may primarily be useful for solid phase volumes in the range of 1-1 000 nL, such as 1-200 nL or 1-50 nL or 1-25 nL. Residence time refers to the time it takes for a liquid aliquot to pass the solid phase in the reaction cavity. Optimization typically will require experimental testing on each particular system in order to reach the acceptable sensitivities (slopes in response curve), dynamic ranges, limits of detection, coefficient of variations etc within the limits discussed above.

The liquid flow through the solid phase can be driven by in principle any kind of force, e.g. electrokinetically or non-electrokinetically created forces. Centrifugal force possibly combined with capillary force are preferred. See further under the heading “Microfluidic devices”.

### Step (iv). Quantification.

This step comprises measuring the amount of detectable analogue bound to BS on the solid phase after step (iii) and relating the measured value to the amount of analyte in an original sample. In the case the detectability of the analogue is caused by the presence of a signal-generating group the signal is measured, for instance as fluorescence, radioactivity, luminescence including chemiluminescence or bioluminescence etc. In the case detectability is based on an affinity group this group is measured by the use of an affinity reactant that is a conjugate between an affinity counterpart to the detectable affinity group of the analyte analogue and a second detectable group that is different from the detectable group of the detectable analogue. This second detectable groups is preferably a signal-generating group typically in the form of a label and selected among the signal-generating groups given above. This latter affinity reactant (conjugate) is typically allowed to be captured to the solid phase during flow conditions as described for the analyte/analyte-related entity or the detectable analyte analogue.

The measurement may comprise that the detectable group on the detectable analyte analogue captured to the solid phase in step (iii) is measured directly on the solid phase in the reaction microcavity (104a-h). In an alternative variant the detectable group is used for formation of a soluble and detectable entity that is transported downstream in the microchannel structure where it is separately measured. See for instance WO 02075312 (Gyros AB).

The measured value obtained for the amount of detectable analogue bound to the solid phase in step (iii) is then related to the amount of analyte in an original sample from which sample 1 derives and/or to the amount of analyte/analyte-related entity in sample 1 or in any intermediary sample obtained during the transformation of the original sample to liquid sample 1. This is typically done according known principles by comparing the measured value with values that have been obtained for one or more standard samples. Typically standard samples comprise a) a series of one, two or more samples containing various known amounts of the analyte, b) one or more samples obtained at an earlier occasion for instance from the same or a different individual or source, c) one or more samples obtained from healthy individuals or from individuals having a particular disease state, etc. The quantification is typically absolute. It may also be relative, e.g. relative to some kind of constituents of sample 1 or of the original sample, relative to another sample taken at an earlier or later occasion and/or from the same or another individual etc.

### Optimizing Response Levels of Liquid Sample Containing Known Amounts F Analyte.

According to the invention this kind of optimisation can be accomplished by performing steps (i)-(iv) twice, thrice or more times with different amounts of analyte or analyte-related entity provided in step (ii) and using a separate microchannel structure for each such run. Different amounts in this context may be accomplished by providing liquid sample 1 as a portion that differs in volume for the different runs/microchannel structures but derives from a common original sample. Each portion typically has been pre-processed in the same manner and thus in most cases therefore contains the same concentration of analyte or analyte-related entity. The different portions/amounts may be accomplished by using microchannel structures which differ with respect to the size of the volume-metering microcavity of the volume-defining unit used for defining the volume of liquid sample 1. Alternatively a microchannel structure that is identical with the first microchannel structure may be used and step (ii) divided into repetitive substeps as discussed elsewhere in this specification thereby providing liquid sample 1 in two or more portions. In the case the structure comprises no appropriate volume-defining unit one can simply introduce sample liquid 1 of different volumes via an inlet port of each structure to be used. The largest amount typically differ from smallest amount with a factor that is >1, such as >1.25 with an upper limit that may be above or below 10 or 100 or 1 000 or 10 000 or higher. The amounts and/or volume of liquid sample 1/analyte/analyte-related entity provided in step (ii) for this kind of series of runs may thus be an multiple 1, 2, 3 etc of the smallest amount used in the series.

As outlined in the claims this variant of the invention comprises that steps (i)-(iv) are carried out at least twice, each time in a separate microchannel structure and with liquid sample 1 as a portion that differ with respect to content of analyte or analyte-related entity. The different amounts is typically accomplished by providing liquid sample 1 in different volumes for the runs. In a preferred variant liquid sample 1 is provided in step (ii) as a series of portions 1¹, 1² . . . 1ⁿ that are serially flowed through the reaction microcavity. There may be intervening washing steps between the portions. n is an integer 1 or larger, such as in the interval of 1-10, preferably ≦4, and different between the microchannel structures used. The portions typically are of the same volume and contains the same amount of analyte or analyte-related entity.

In the case the microfluidic device comprises a plurality of the microchannel structures, the runs described for this variant may be carried out on the same microfluidic device, for instance with at least the flowing in step (ii) or in step (iii) in parallel for the structures used.

Alternatively, a similar optimisation may be accomplished by utilizing solid phases with different loadings of the analyte counterpart in the different runs above (while then loading the same amount of analyte or analyte-related entity in step (ii)). Different loadings of the analyte counterpart is preferably accomplished by the use of an immobilizing pair, such as an immobilizing affinity pair, as discussed above and then include a deficient amount of the analyte counterpart exhibiting RS_{cp} or a mixture of the RS_{cp} analyte counterpart and a nonsense reactant exhibiting the group RSₙₛ in the liquid aliquot that is used as a carrier for presenting the porous bed exhibiting RSₛₚ for these reactants.

### Microfluidic Devices

A suitable microfluidic device for use in the innovative method comprises one, two or more microchannel structures (101a-h) in which one or more liquid aliquots/samples that have volumes in the μL-range, typically in the nanolitre (nL) range, containing various kinds of reactants, such as analytes and reagents, products, samples, buffers and/or the like are processed, e.g. to obtain liquid samples 1 and 2. The μL-range means volumes ≦1 000 μL, such as ≦100 μL or ≦10 μL and includes the nL-range that has an upper end of 5 000 nL but in most cases relates to volumes ≦1 000 nL, such as ≦500 nL or ≦100 nL. The nL-range includes the picolitre (pL) range. Sample aliquots containing an analyte or an analyte-related entity and in particular as introduced through an inlet port are typically in the μL-range with volumes above 1000 mL which does not eclude that they may be in the nL-range.

Corresponding aliquots containing a reagent such as the detectable analyte analogue are typically in the nL-range as given above although volumes in the μL-range that are ≧1 000 μL may be used. A microchannel structure (101a-h) comprises one or more cavities and/or conduits that have a cross-sectional dimension that is ≦10³ μm, preferably ≦5×10² μm, such as ≦102 μm.

A microchannel structure (101a-h) of the microfluidic device thus may comprise one, two, three or more functional units selected among: a) inlet arrangements (102,103a-h) comprising for instance an inlet port/inlet opening (105a-b,107a-h), possibly together with a volume-defining unit (106a-h,108a-h), b) microconduits for liquid transport, c) reaction microcavities (104a-h); d) mixing microcavities/units; e) units for separating particulate matters from liquids (may be present in the inlet arrangement), f) units for separating dissolved or suspended components in the sample from each other, for instance by capillary electrophoresis, chromatography and the like; g) detection microcavities; h) waste conduits/microcavities (112,115a-h); i) valves (109a-h,110a-h); j) vents (116a-i) to ambient atmosphere; liquid splits (liquid routers) etc. A functional unit may have more than one functionality, e.g. a reaction microcavity (104a-h) and a detection microcavity may coincide. Various kinds of functional units in microfluidic devices have been described by Gyros AB/Amersham Pharmacia Biotech AB: WO 99055827, WO 99058245, WO 02074438, WO 02075312, WO 03018198 (US 20030044322), WO 03034598, WO 05032999, WO 04103890, PCT/SE2005/000403 and by Tecan/Gamera Biosciences: WO 01087487, WO 01087486, WO 00079285, WO 00078455, WO 00069560, WO 98007019, WO 98053311.

In advantageous forms a reaction microcavity (104a-h) intended for a hydrophilic porous bed is connected to one or more inlet arrangements (upstream direction) (102,103a-h), each of which comprises an inlet port (105a-b,107a-h) and at least one volume-defining unit (106a-h,108a-h). An advantageous inlet arrangement (103a-h) comprises that the arrangement is only connected to one microchannel structure (101a-h) and/or reaction microcavity (104a-h) intended to contain the solid phase material. Another advantageous inlet arrangement (102) comprises that the arrangement is common to all or a subset (100) of the microchannel structures (101a-h) and/or reaction microcavities (104a-h) intended to contain the solid phase material. This latter variant comprises a common inlet port (105a-b) and a distribution manifold with one volume-defining unit (106a-h) for each microchannel structure/reaction microcavity (101a-h/104a-h) of the subset (100). In both variants, a volume-defining unit (106a-h,108a-h) is communicating with downstream parts of a microchannel structure (101a-h), e.g. with the reaction microcavity (104a-h). Each volume-defining unit (106a-h,108a-h) typically has a valve (109a-h,110a-h) at its outlet end. This valve is typically passive, for instance utilizing a change in chemical surface characteristics at the outlet end, such as a boundary between a hydrophilic and hydrophobic surface (hydrophobic surface break) (WO 99058245 (Amersham Pharmacia Biotech AB)) and/or in geometric/physical surface characteristics, such as an abrupt change in a lateral cross-dimension of the inner surface/wall (WO 98007019 (Gamera)).

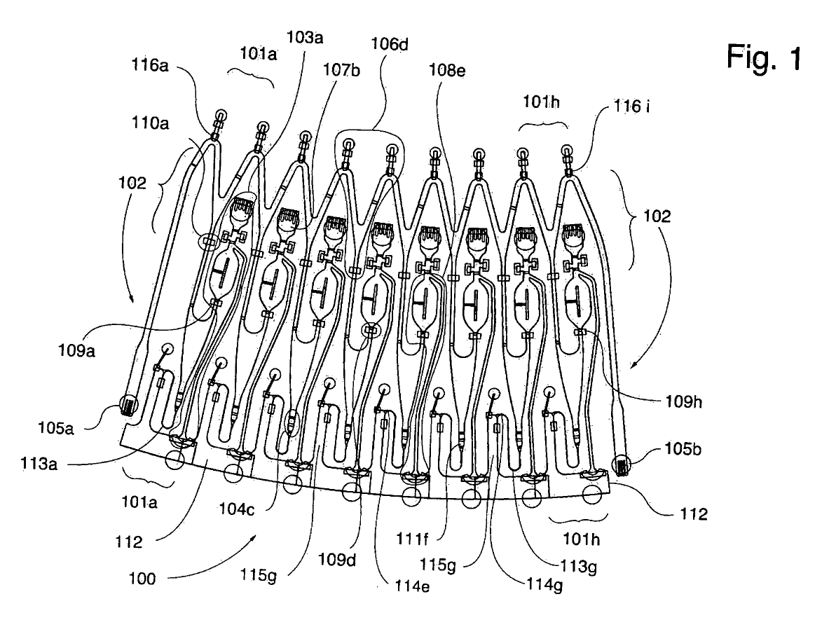

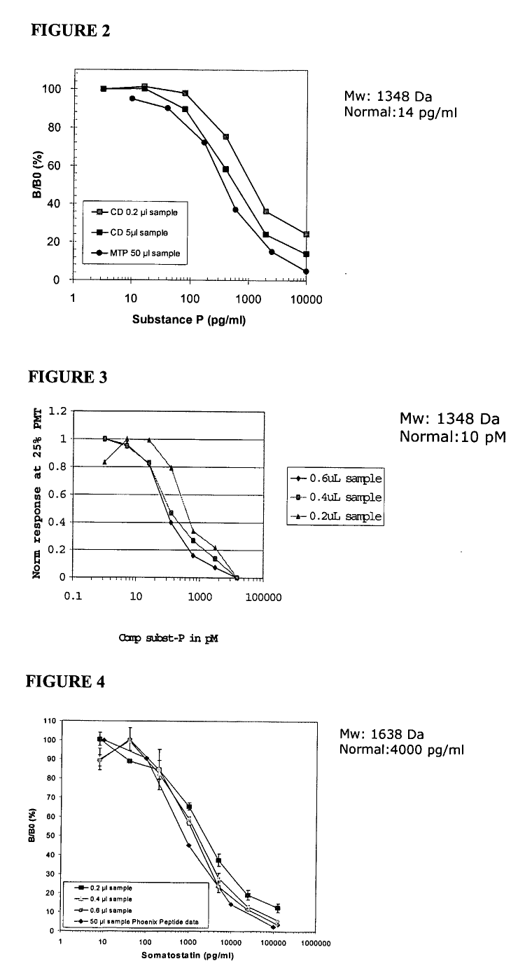

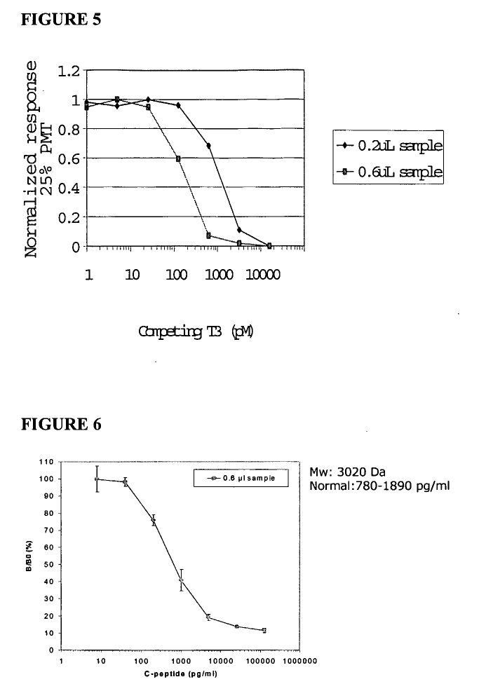

Typical inlet arrangements with inlet ports, volume-defining units, distribution manifolds, valves etc have been presented in WO 02074438, WO 02075312, WO 02075775 and WO 02075776 (all Gyros AB). As illustrated in these publications and in FIG. 1 the preferred volume-defining units are based on the overflow principle to get rid of excess liquid. This typically means that there is an overflow microconduit at the inlet end of a volume-metering microcavity of each unit and that this microcavity is preferably constricted at its junction with the overflow microconduit, i.e. at the inlet end of the volume-metering microcavity. Suitable volume-defining units for on-device metering/defining of liquid aliquots containing a reactant used in the innovative method should be capable of metering with accuracy within ±25% such as within ±15% or within ±10% or within ±6% or within ±3%. This in particular applies if the reactant is an analyte or an analyte-related entity but is typically also applicable to other reactants, such as an analyte counterpart, a detectable analyte analogue, one or more other reactants needed for the measurement in step (iii) etc.

The mixing functions if present are typically based on

a) mixing in a mixing microconduit, and/or
b) collecting the two liquid samples in a separate mixing microcavity and cause mixing by
i) a mechanical mixer e.g. by including magnetic particles in the mixing microcavity, and/or
ii) the use of inertia force possible enforced by including magnetic particles in the microcavity combined with external magnets, and/or
iii) back and forth transport in a microconduit connected to the mixing microcavity.

Suitable inertia force may be created by accelerating and/or decelerating movements of the device, for instance by spinning back and forth. Back and forth transport may be caused by using capillary transport in one direction and centrifugal force in the opposite direction, i.e. intermittent spinning of the appropriate microfluidic device. The outlet of the mixing microcavity is typically equipped with a valve, such as a non-closing valve for instance a passive valve defined by a hydrophobic break or an abrupt change in a lateral cross-dimension of the inner surface/wall of a microconduit of a microchannel structure (101a-h). One or both of the inlet ports may or may not be equipped with a volume-defining unit as described in the previous paragraph. Mixing as described above has been presented in U.S. Pat. No. 6,582,662 (Tecan); U.S. Pat. No. 6,572,432 (Tecan), US 20020025583 (First Medical Inc), WO 02074438 (Gyros AB), WO 03018198, WO 05032399 (Gyros AB), PCT/SE2005/000403 (Gyros AB), U.S. Pat. No. 5,591,643 (Abaxis), U.S. Ser. No. 60/634,657 and corresponding SE 04030030-0) etc.

Each microchannel structure (101a-h) has at least one inlet opening (105a-b,107a-h) for liquids and at least one outlet opening for excess of air (vents) (116a-i) and possibly also for liquids (circles in the waste channel (112)).

The microfludic device contains a plurality of microchannel structures/device intended to contain the BS solid phase. Plurality in this context means two, three or more microchannel structures (101a-h) and typically is ≧10, e.g. ≧25 or ≧90 or ≧180 or ≧270 or ≧360.

Different principles may be utilized for transporting the liquid within the microfluidic device/microchannel structures (101a-h) between two or more of the functional units. Inertia force may be used, for instance by spinning the disc as discussed in the subsequent paragraph. Other useful forces are capillary forces, electrokinetic forces, non-electrokinetic forces such as capillary forces, hydrostatic pressure etc.

The microfluidic device typically is in the form of a disc. The preferred formats have an axis of symmetry (Cₙ) that is perpendicular to or coincides with the disc plane, where n is an integer ≧2, 3, 4 or 5, preferably ∞ (C_{∞}). In other words the disc may be rectangular, such as square-shaped and other polygonal forms but is preferably circular. Spinning the device around a spin axis that typically is perpendicular or parallel to the disc plane may create the necessary centrifugal force. Variants in which the spin axis is not perpendicular to a disc plane are given in WO 04050247 (Gyros AB).

If centrifugal force is used for driving liquid flow through the reaction microcavity/solid phase, the reaction microcavity (104a-h) is typically oriented with the flow direction essentially radially outwards from the spin axis.

The preferred devices are typically disc-shaped with sizes and/or forms similar to the conventional CD-format, e.g. sizes that are in the interval from 10% up to 300% of a circular disc with the conventional CD-diameter (12 cm).

Microchannels/microcavities of a microfluidic device may be manufactured from an essentially planar substrate surface that exhibits one or more channels and/or cavities of a microchannel structure in uncovered form. The uncovered cahnnel(s) and/or cavity(ies) are in a subsequent step covered by another essentially planar substrate (lid). See WO 91016966 (Pharmacia Biotech AB) and WO 01054810 (Gyros AB). Both substrates are preferably fabricated from plastic material, e.g. plastic polymeric material.

The fouling activity and hydrophilicity of inner surfaces should be balanced in relation to the application. See for instance WO 0147637 (Gyros AB).

The terms “wettable” (hydrophilic) and “non-wettable” (hydrophobic) in the context of an inner surface of a microfluidic device means that the surface has a water contact angle ≦90° or ≧90°, respectively. In order to facilitate efficient transport of a liquid between different functional parts, inner surfaces of the individual parts should primarily be wettable, preferably with a contact angle ≦60° such as ≦50° or ≦40° or ≦30° or ≦20°. These wettability values apply for at least one, two, three or four of the inner walls of a microconduit. In case one or more of the inner walls have a higher water contact angle this can be compensated for by a lower water contact angle for the inner wall(s). The wettability, in particular in inlet arrangements should be adapted such that an aqueous liquid will be able to fill up an intended microcavity/microconduit by capillarity (self suction) once the liquid has started to enter the cavity/microconduit. A hydrophilic inner surface in a microchannel structure (101a-h) may comprise one or more local hydrophobic surface breaks in a hydrophilic inner wall, for instance for introducing a passive valve, an anti-wicking means, a vent solely function as a vent to ambient atmosphere etc (rectangles in FIG. 1). See for instance WO 99058245, WO 02074438, US 20040202579, WO 2004105890, WO 2004103891 (all Gyros AB).

Contact angles refer to values at the temperature of use, typically +25° C., are static and can be measured by the method illustrated in WO 00056808 and WO 01047637 (all Gyros AB).

### EXPERIMENTAL PART

### Microfluidic Devices

The microfluidic devices used were circular (CD) and adapted for transportation of liquid by centrifugal force by spinning as discussed above (for passage of passive valve functions and/or porous beds). Each microchannel structure contained the appropriate volume-defining units for the liquid volumes introduced. Mainly two different microfluidic devices were used: one with a microchannel structure that has two different volume-defining units each of which enabled on-device metering of 0.2 μL aliquots (FIG. 1) and the other one with a microchannel structure that has one volume-defining unit enabling on-device metering of 1 μL aliquots and another volume-defining unit enabling on-device metering of 5 μL aliquots.

The immobilizing pairs in the columns were streptavidin and biotin (example 1) and goat anti rabbit IgG antibody and a rabbit IgG antibody (examples 2-5).

In addition to the washing step expressly indicated in examples 1-5 there were also washing step included between aliquots containing a reactant.

### Instrumentation

The microfluidic devices (CDs) were processed in an automated system Gyrolab Workstation (Gyros AB, Uppsala, Sweden) equipped with a Laser Induced Fluorescence (LIF) module (examples 2-5) and a luminometer (example 1). The system also is equipped with a CD-spinner, holder for microtiter plates (MTP), a robotic arm with a holder for 10 capillaries connected to 5 syringe pumps, etc.

The Gyrolab Workstation is a fully automated robotic system controlled by application-specific software. An application specific method within the software controls the spinning of the CD at the precisely controlled speeds and thereby controls the movement of liquids through the microstructures as the application proceeds. Special software was included in order to reduce background noise.

See also WO 02075312 (Gyros AB), WO 03025548 and US 20030054563 (Gyros AB), WO 03025585 and US 200030055576 (Gyros AB), WO 03056517 and US 200301156763 (Gyros AB) and also www.gyros.com.

### Example 1

### Inhibition Assay of Substance P

Goat anti rabbit IgG (H+L) DS Grade (Zymed) was biotinylated with EZ-LinkTMSulfo-NHS-LC-Biotin (PIERCE, Rockford, Ill.; USA) according to the manufacturers instructions. The biotinylated goat anti rabbit IgG (0.1 mg/mL; 0.2 μL or 1 μL) was loaded to the streptavidin columns in the CD. Subsequently rabbit anti Substance P antibody (0.2 μL or 1 μL) (Assay Designs) was loaded to the columns. Sample standards (0.2 μL or 5 μL) containing Substance P were passed over the columns. ALP-Substance P conjugate (Assay Designs) was then added to the columns. After repeated (3 times) wash of the columns with PBST buffer, alkaline phosphatase substrate (CDP-Star (0.8 mM)+Emerald-II™ (20% (v/v))(Applied Biosystems, Bedford, Mass., USA) was passed over the column. After 10 minutes of incubation, luminescence signals were recorded from the columns by means of the luminometer. Normal serum concentrations of Substance P (1348 Da) are expected to be 14 pg/mL.

### Example 2

### Inhibition Assay of Substance P

F(ab₂)′fragments of goat anti rabbit IgG (Jackson ImmunoReaearch prod.#111-006-0146) were coupled to 10 μm particles (TSKgel Tresyl-5PW, TOSOH BIOSCIENCE, Stuttgart, Germany) according to the manufacturers instructions. These particles were packed as columns in the CD. Rabbit anti Substance P (0.2 μL) (Assay Designs) was loaded to the columns. Sample standards (0.2 μL) containing Substance P were passed over the columns. Data in the graph illustrate results obtained with the samples introduced once (1×0.2 μL), twice (2×0.2 μL) or three (3×0.2 μL) times into the CD. Biotinyl-Substance P (150 pM) (Phoenix Peptide GmbH, Karlsruhe, Germany, Prod.#B-061-06) was then added to the columns. After repeated (3 times) wash of the columns with PBST buffer, streptavidin-Alexa647 conjugate (0.5 nM) (Molecular Probes) was passed over the column. After additional four washes of the columns with PBST, signals were recorded in the columns by means of a laser induced fluorescence (LIF) detector in the Gyrolab workstation (Gyros AB). Normal serum levels of Substance P (1348 Da) are expected to be 10 pM

### Example 3

### Inhibition Assay of Somatostatin

Reagents were used from the Somatostatin EIA kit (Phoenix Peptide GmbH, Karlsruhe, Germany Prod.#EK-060-03). F(ab₂)′fragments of goat anti rabbit IgG (Jackson ImmunoReaearch prod.#111-006-0146) were coupled to 10 μm particles (TSKgel Tresyl-5PW, TOSOH BIOSCIENCE, Stuttgart, Germany) according to the manufacturers instructions. These particles were packed as columns in the CD. Rabbit anti Somatostatin (0.2 μL) (Phoenix Peptide GmbH, Karlsruhe, Germany) was loaded to the columns. Sample standards (0.2 μL) containing Somatostatin (Phoenix Peptide GmbH, Karlsruhe, Germany) were passed over the columns. Data in the graph illustrate results obtained with the samples introduced once (1×0.2 μL), twice (2×0.2 μL) or three (3×0.2 μL) times into the CD. Biotinyl-Somatostatin (2× concentration compared to protocol for MTP assay; Phoenix peptide Somatostatin EIA kit Prod.#EK-060-03) was then added to the columns. After repeated (3 times) wash of the columns with PBST buffer, streptavidin-Alexa647 conjugate (0.5 mM) (Molecular Probes) was passed over the column. After additional four washes of the columns with PBST, signals were recorded in the columns by means of laser induced fluorescence (LIF) detector in Gyrolab workstation (Gyros AB). Normal serum levels of Somatostatin (Mw=1638 Da) are expected to be 4000 pg/mL.

### Example 4

### Inhibition Assay of T3

F(ab₂)′fragments of Goat anti rabbit IgG (Jackson ImmunoReaearch prod.#111-006-0146) were coupled to 10 μm particles (TSKgel Tresyl-5PW, TOSOH BIOSCIENCE, Stuttgart, Germany) according to the manufacturers instructions. These particles were packed as columns in the CD. Monoclonal mouse anti T3 (0.2 μL; 50 pM) (Abcam Prod.#Ab 1981-1) was loaded to the columns. Sample standards (0.2 μL) containing T3 were passed over the columns. Data in the graph illustrate results obtained with the samples introduced once (1×0.2 μL) or three (3×0.2 μL) times into the CD. Biotinyl-BSA-T3 (0.2 μL; 1 nM) was then added to the columns. After repeated (3 times) wash of the columns with PBST buffer, streptavidin-allophycocyanin conjugate (1 nM) (Molecular Probes) was passed over the column. After additional four washes of the columns with PBST, signals were recorded in the columns by means of laser induced fluorescence (LIF) detector in Gyrolab workstation (Gyros AB). The sensitivity in the assay (IC_{50 [}50% of max signal] is lowered från 1200 pM to 200 pM) with the sample addition compared to one sample addition.

### Example 5

### Inhibition Immunoassay of C-Peptide

Reagents were used from C-peptide (human) EIA kit (Phoenix Peptide GmbH, Karlsruhe, Germany Prod. #EK-035-01). F(ab₂)′fragments of goat anti rabbit IgG (Jackson ImmunoReaearch prod.#111-006-0146) were coupled to 10 μm particles (TSKgel Tresyl-5PW, TOSOH BIOSCIENCE, Stuttgart, Germany) according to the manufacturers instructions. These particles were packed as columns in the CD. Rabbit anti C-peptide (0.2 μL) (Phoenix Peptide GmbH, Karlsruhe, Germany) was loaded to the columns. Sample standards (0.2 μL) containing C-peptide (Phoenix Peptide GmbH, Karlsruhe, Germany) were passed over the columns. Data in the graph illustrate results obtained with the samples introduced three (3×0.2 μL) times into the CD. Biotinyl-C-peptide (2× concentration compared to protocol for MTP assay; Phoenix peptide Somatostatin EIA kit Prod.#EK-035-01) was then added to the columns. After repeated (3 times) wash of the columns with PBST buffer, Steptavidin-Alexa647 conjugate (0.5 nM) (Molecular Probes) was passed over the column. After additional four washes of the columns with PBST, signals were recorded in the columns by means of laser induced fluorescence (LIF) detector in Gyrolab workstation (Gyros AB). Normal serum levels of C-peptide (Mw=3020 Da) are expected to be 780-1890 pg/mL.

Certain innovative aspects of the invention are defined in more detail in the appending claims. Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for the determination of an analyte in an original liquid sample by performing an inhibition affinity assay in a microchannel structure of a microfluidic device, comprising the steps of:
(i) providing the microfluidic device in a form where the microchannel structure comprises a reaction microcavity which a) an inlet end, and b) contains a solid phase with binding sites (BS) for an analyte or an analyte-related entity,
(ii) providing a liquid sample 1 containing the analyte or the analyte-related entity within the microchannel structure at the inlet end of the reaction microcavity, and flowing sample 1 through the reaction microcavity under conditions such that the analyte or the analyte-related entity is captured by BS leaving a portion of BS unoccupied (free BS),
(iii) providing a liquid sample 2 which contains an analytically detectable analogue of the analyte or of the analyte-related entity, and flowing sample 2 through said reaction microcavity to capture the analogue by free BS, and
(iv) measuring the amount of analyte analogue captured in step (iii) and relating this amount to the amount of analyte in an original sample.

2. The method of claim 1, wherein the microchannel structure comprises one or more inlet ports each of which contains a volume-defining unit.

3. The method of claim 2, wherein the analyte, the analyte-related entity and/or the analogue dissolved in a liquid are/is introduced into the microchannel structure via such an inlet port, “hereafter an aliquot of such a liquid is volume-defined in the volume-defining unit associated with the inlet port used and further processed and/or transported to the inlet end of the reaction microcavity to be used for step (ii) or step (iii).

4. The method of claim 1, wherein BS provided in step (i) are in molar excess compared to the amount of the analyte or the analyte-related entity provided in liquid sample 1 in step (ii).

5. The method of claim 1, wherein the molar amount of the analyte or of the analyte-related entity captured in step (ii) constitutes in the range of 5-95% of the molar amount of BS provided in step (i).

6. The method of claim 1, wherein the amount of said detectable analogue is in excess compared the amount of free BS after step (ii), preferably compared the total amount of BS provided in step (i).

7. The method of claim 1 wherein, BS is part of an affinity: counterpart to the analyte that has been immobilized to the solid phase by the use of an immobilizing binding pair of reactive structures RSₛₚ and RS_{cp} that before immobilization are present on the solid phase and the counterpart, respectively, and during immobilization are capable of reacting with each other to the formation of a bond that that links the counterpart to the solid phase and resists undesired cleavage during the method.

8. The method of claim 7, wherein the immobilizing binding pair is capable of forming a covalent bond.

9. The method of claim 7, wherein said immobilizing binding pair is selected among immobilizing affinity pairs.

10. The method of claim 7 wherein step (i) comprises the steps of: a) providing the reaction microcavity with a solid phase that is in a form exhibiting the reactive structure RSₛₚ but no BS, and b) providing a liquid sample 3 containing the counterpart at the inlet end of the reaction microcavity and flowing sample 3 through the microcavity under conditions such that the counterpart will become immobilized to the solid phase at least in a zone close to the inlet end.

11. The method of claim 10, wherein liquid sample 3 also contains a nonsense reactant that exhibits a reactive structure RSₙₛ, that is capable of reacting with RSₛₚ during the same conditions as RS_{cp} and typically is equal to RS_{cp}.

12. The method of claim 1, wherein
a) the microchannel structure comprises one or more inlet ports each of which contains a volume-defining unit, and
b) said counterpart and/or said nonsense reactant, if present, are/is introduced dissolved in a liquid via such an inlet port whereafter an aliquot of such a liquid is defined in a volume-defining unit associated with the inlet port used and thereafter processed and/or transported to the inlet end of the reaction microcavity.

13. The method of claim 1, wherein the centrifugal force and/or capillary force is utilized for transporting liquid through at least a part of the microchannel structure.

14. The method of claim 1, wherein the method comprises that steps (i)-(iv) are carried out at least twice, each time in a separate microchannel structure and with the volume of liquid sample 1 being different between the times including the same relative difference for the amount of the analyte or the analyte-related entity as between the volumes of liquid sample 1.

15. The method of claim 14, wherein the liquid sample 1 is provided in step (ii) as a series of portions 1\I² . . . 1ⁿ in that are serially flowed through the reaction microcavity possibly with intervening washing steps, where n is an integer 1 or larger, such as in the interval of 1-10, preferably <4, and different between the microchannel structures used.

16. The method of claim 14, wherein the largest amount of said different amounts a) differs from the smallest amount with a factor within the range of >1 or b) is an even multiple of the smallest amount.

17. The method of claim 14, wherein the microfluidic device comprises a plurality of said microchannel structure and that at least two of said plurality of microchannel structures are utilized for performing said at least twice of the steps (i) (iv).

18. The method of claim 17, wherein the flowing of a corresponding liquid sample through the reaction microcavity are carried out in parallel for the microchannel structures used for two or more of said at least two times.
